(19) United States
(12) Patent Application Publication
(10) Pub. No.: US 2025/0132048 A1
(43) Pub. Date: Apr. 24, 2025

(54) PREDICTION DEVICE, METHOD OF GENERATING PREDICTION MODEL, AND COMPUTING DEVICE

(71) Applicant: NIHON KOHDEN CORPORATION, Tokyo (JP)

(72) Inventors: Takuya KAWASHIMA, Tokorozawa-shi (JP); Daisuke HORIGUCHI, Tokorozawa-shi (JP)

(73) Assignee: NIHON KOHDEN CORPORATION, Tokyo (JP)

(21) Appl. No.: 18/828,428

(22) Filed: Sep. 9, 2024

(30) Foreign Application Priority Data
Oct. 18, 2023 (JP) 2023-179592

(51) Int. Cl. G16H 50/30; G06F 17/17

(52) U.S. Cl. CPC G16H 50/30; G06F 17/17

(57) ABSTRACT

An interface receives time series data including multiple observed values of an observed parameter that are acquired at different time points for obtaining physiological information of a subject. A processor inputs the time series data into a prediction model to output, as a prediction result, a range within which values interpolating the multiple observed values may fall. The processor generates at least one first interpolated value by interpolating the multiple observed values with a first method. The processor generates at least one second interpolated value by interpolating the multiple observed values with a second method that is different from the first method. The processor identifies, as the prediction result, a range defined between a maximum value and a minimum value among the first interpolated value and the second interpolated value.

### CROSS-REFERENCE TO RELATED APPLICATION

The present application is based on Japanese Patent Application No. 2023-179592 filed on Oct. 18, 2023, the entire contents of which are incorporated herein by reference.

### BACKGROUND

The presently disclosed subject matter relates to a prediction device configured to input, to a prediction model, time series data including multiple observed values of an observed parameter acquired from a subject for obtaining physiological information of the subject, thereby predicting an available range of a value interpolating the observed values. The presently disclosed subject matter also relates to a method of generating the prediction model as well as a computing device configured to generate the prediction model.

Inferential models that are generated with machine learning techniques to replace or support clinical decisions of medical workers in the field of medical clinical practice are known. For example, Non-patent Document 1 discloses a model configured to predict specific physiological information based on time series data of an observed parameter. Examples of specific physiological information include a remaining length of stay in an intensive care unit (ICU), and the like. Non-patent Document 1 reports that the prediction model exhibits excellent performance with respect to an observed parameter such as a heart rate that would be frequently acquired, whereas the prediction model does not exhibit good performance with respect to an observed parameter such as the number of leukocytes that would be intermittently acquired (difficult to acquire a sufficient amount of time series data).
Non-patent Document 1: G. Harerimana, et al. “A Multi-Headed Transformer Approach for Predicting the Patient's Clinical Time-Series Variables From Charted Vital Signs”, IEEE Access (Volume 10), 105993-106004, Oct. 3, 2022

### SUMMARY

It is demanded to suppress degradation of prediction accuracy of a prediction model configured to use, as an input, a value interpolating observed values acquired in an environment in which time series data of an observed parameter are not stably acquired.

A first illustrative aspect of the presently disclosed subject matter provides a prediction device, comprising:
an interface configured to receive time series data including multiple observed values of an observed parameter that are acquired at different time points for obtaining physiological information of a subject; and
a processor configured to input the time series data into a prediction model to output, as a prediction result, a range within which values interpolating the multiple observed values may fall,
wherein the processor is further configured to:
   generate at least one first interpolated value by interpolating the multiple observed values with a first method;
   generate at least one second interpolated value by interpolating the multiple observed values with a second method that is different from the first method; and
   identify, as the prediction result, a range defined between a maximum value and a minimum value among the first interpolated value and the second interpolated value.

In general, the shorter the time interval for acquiring multiple observed values of the observed parameter, the less the necessity for interpolating an estimated value between the multiple observed values. As a result, it is possible to suppress degradation of the prediction model that uses the time series data of the observed parameter as the input. On the other hand, for example, the axillary temperature is generally measured at a frequency of once every several hours in a clinical practice, and there may be a case where it is difficult to secure time series data including a sufficient number of observed values with respect to the observed parameter. However, according to the configuration of the first illustrative aspect, a range defined by the maximum value and the minimum value selected from multiple interpolated values generated with multiple different methods is specified as a reasonable range within which the interpolated values may finally fall. As a result, it is possible to suppress a bias in the interpolation tendency that may be caused by relying on a specific interpolation method. Accordingly, it is possible to suppress degradation of prediction accuracy of a prediction model configured to use, as an input, a value interpolating observed values acquired in an environment in which time series data of an observed parameter are not stably acquired.

A second illustrative aspect of the presently disclosed subject matter provides a prediction device, comprising:
an interface configured to receive time series data including multiple observed values of an observed parameter that are acquired at different time points for obtaining physiological information of a subject; and
a processor configured to input the time series data into a prediction model to perform at least one prediction of a range within which values interpolating the multiple observed values may fall,
wherein the processor is further configured to select, as the prediction model, one of multiple prediction models in accordance with a time interval between the different time points; and
wherein the multiple prediction models are generated by machine learning so as to correspond to different time intervals between the different time points.

In general, in a case where a time interval for acquiring multiple observed values of an observed parameter is constant, it is possible to suppress degradation of the accuracy of prediction performed by a prediction model for a range within which unobserved values to be interpolated between the multiple observed values may fall. On the other hand, since it is general that the axillary temperature, for example, is manually measured by a medical worker, there may be a case where the time interval for acquiring observed values of an observed parameter is not constant. However, according to the configuration of the second illustrative aspect, since the prediction of values to be interpolated is performed by selecting an adequate one of multiple prediction models generated by multiple machine learnings that are made different in correspondence with the time interval, it is possible to suppress degradation of the prediction accuracy of the prediction model that uses, as an input, values interpolated between observed values in the case where the time interval for acquiring the observed values is not constant.

A third illustrative aspect of the presently disclosed subject matter provides a method of generating a prediction model adapted to predict physiological information with a computing device, the method comprising:
acquiring observation data including multiple observed values of an observed parameter acquired from a living body with a first time interval for obtaining the physiological information;
downsampling at least one of the multiple observed values to generate training data including multiple observed values provided with a second time interval that is longer than the first time interval;
performing supervised learning with the training data such that the multiple observed values as downsampled are regarded as a ground truth;
configuring the prediction model so as to predict, with respect to multiple observed values of the observed parameter acquired from a living body with the second time interval as an input, a range within which at least one value interpolating the multiple observed values as inputted may fall; and
generating another training data from the observation data while changing the second time interval.

A fourth illustrative aspect of the presently disclosed subject matter provides a computing device configured to generate a prediction model adapted to predict physiological information, the computing device comprising:
an interface configured to receive observation data including multiple observed values of an observed parameter acquired from a living body with a first time interval for obtaining the physiological information; and
a processor configured to:
   downsample at least one of the multiple observed values to generate training data including multiple observed values provided with a second time interval that is longer than the first time interval;
   perform supervised learning with the training data such that the multiple observed values as downsampled are regarded as a ground truth;
   configure the prediction model so as to predict, with respect to multiple observed values of the observed parameter acquired from a living body with the second time interval as an input, a range within which at least one value interpolating the multiple observed values as inputted may fall; and
   generate another training data from the observation data while changing the second time interval.

By performing the machine learning with the training data in which the second time interval is so determined as to match with the time interval between multiple observed values included in the time series data acquired from the subject, the prediction model as generated can obtain capability of specifying, with at least one prediction, an appropriate range within which unobserved values to be interpolated between the observed values acquired with that time interval may fall. In a case where the time interval between the multiple observed values included in the time series data is not constant, machine learning with training data in which the second time interval is varied may be performed to provide multiple prediction models. As a result, it is possible to configure a prediction device capable of estimate, with at least one prediction, a range within which unobserved values to be interpolated between multiple observed values acquired with each of different time intervals may fall.

### DESCRIPTION OF THE DRAWINGS

FIG. 1 illustrates a functional configuration of a prediction system according to one exemplary embodiment.

FIG. 2 illustrates an exemplary flow of processing executed in a prediction device of FIG. 1.

FIG. 3 illustrates another exemplary processing executed in the prediction device of FIG. 1.

FIG. 4 illustrates an exemplary flow of processing executed in the prediction system of FIG. 1.

### DESCRIPTION OF EMBODIMENTS

Exemplary embodiments will be described in detail with reference to the accompanying drawings. In the drawings, the scale is appropriately changed in order to make each element to be described have a recognizable size.

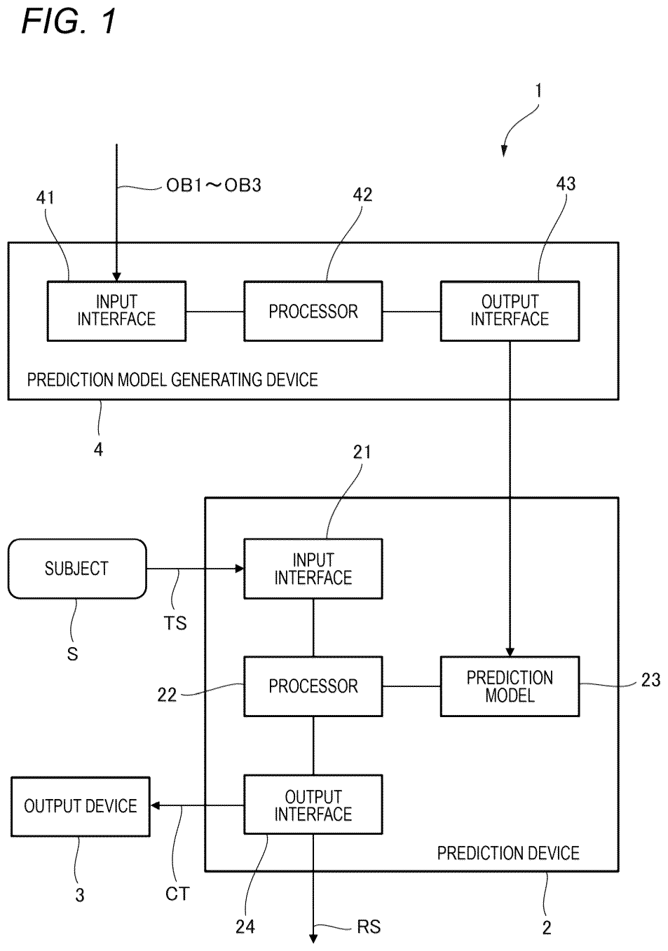

FIG. 1 illustrates a functional configuration of a prediction system 1 according to an exemplary embodiment. The prediction system 1 includes a prediction device 2.

The prediction device 2 according to the present example is a device configured to predict, based on multiple observed values of axillary temperature of a subject S that are acquired at multiple time points, an available range of a value of the axillary temperature that is to be interpolated in a time period defined between the multiple time points. The axillary temperature is an example of an observed parameter. Examples of other observed parameters include a respiratory rate that is obtained by visually observing thoracic motions, a Glasgow Coma Scale (GCS) that is obtained by visually confirming the state of consciousness, a urine volume that is measured in a urine collection bag, an index value that is obtained through a blood test (the number of erythrocytes, the number of leukocytes, a blood lactate value, an arterial oxygen partial pressure, and the like), and a blood pressure that is obtained by non-invasive measurement.

As used herein, the term “time series of an observed parameter” means changes over time of a value of an observed parameter that is acquired at multiple time points. The interval between the multiple time points may be constant or may not be constant.

The prediction device 2 includes an input interface 21. The input interface 21 is configured as a hardware interface adapted to receive the above-described time series data TS. The time series data TS may be inputted from an adequate sensor or measurement device attached to the subject S, or may be inputted from an adequate input device with a user operation. The time series data TS may be in the form of analog data or digital data, in accordance with the specification of the input source. In the case where the time series data TS is in the form of analog data, the input interface 21 includes an adequate conversion circuit including an A/D converter.

The prediction device 2 includes a processor 22 and a prediction model 23. The prediction model 23 is a computer algorithm adapted to be executed by the processor 22. The prediction model 23 is configured to output, based on multiple observed values of axillary temperature of a subject S that are acquired at multiple time points, an available range of a value of the axillary temperature that is to be interpolated in a time period defined between the multiple time points, as a prediction result.

The prediction device 2 includes an output interface 24. The processor 22 is configured to input the time series data TS received by the input interface 21 to the prediction model 23, and configured to output, from the output interface 24, result data RS corresponding to the prediction result outputted from the prediction model 23.

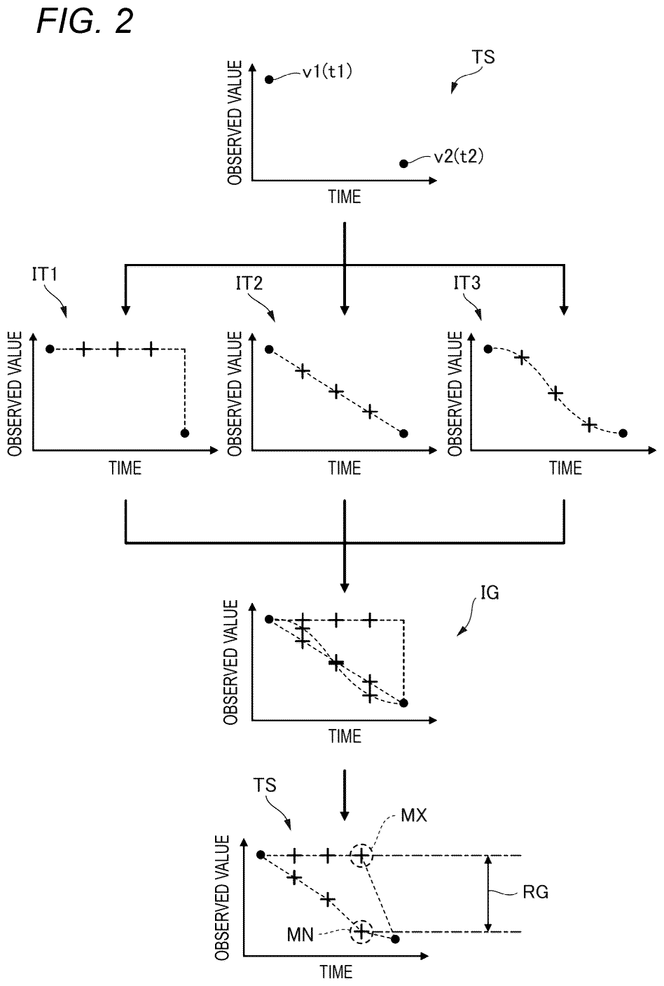

An exemplary specific operation of the prediction model 23 will be described with reference to FIG. 2. The time series data TS received by the input interface 21 include an observed value v1 of the axillary temperature acquired at a time point t1 and an observed value v2 of the axillary temperature acquired at a time point t2.

The processor 22 is configured to generate multiple interpolation data sets in different manners based on the time series data TS. In this example, a first interpolation data set IT1, a second interpolation data set IT2, and a third interpolation data set IT3 are generated.

In the first interpolation data set IT1, three non-invasive blood pressure values are interpolated during a time period between the time points t1 and t2. The interpolation method is based on the LOCF (Last Observation Carried Forward) method. In this method, interpolation is performed such that an observed value at a certain time point is maintained until a next observed value is obtained. The number of interpolated values may be arbitrarily determined.

In the second interpolation data set IT2, three non-invasive blood pressure values are interpolated during a time period between the time points t1 and t2. The interpolation method is based on a linear interpolation method. In this method, a value corresponding to an observed value at an arbitrary time point that is located on a linear line connecting two observed values is interpolated. The number of interpolated values may be arbitrarily determined.

In the third interpolation data set IT3, three non-invasive blood pressure values are interpolated during a time period between the time points t1 and t2. The interpolation method is based on a spline interpolation method. In this method, a value corresponding to an observed value at an arbitrary time point that is located on a polynomial curve connecting two observed values is interpolated. The number of interpolated values may be arbitrarily determined.

As long as the interpolation methods are different from each other, the number of the multiple interpolation data sets to be generated by the processor 22 may be arbitrarily determined. An arbitrary interpolation data set selected from the multiple interpolation data sets as generated may be an example of the first interpolation data set. Similarly, any other interpolation data set selected from the multiple interpolation data sets may be an example of the second interpolation data set. At least one interpolated value included in the first interpolation data set is an example of a first interpolated value. At least one interpolated value included in the second interpolation data set is an example of a second interpolated value.

It should be noted that, as used herein, the expression “different interpolation methods” is not intended to refer to only a case where the types of interpolation methods are different from each other. As an example, an interpolation data set generated by a spline interpolation with a cubic polynomial, and an interpolation data set generated by a spline interpolation with a fifth polynomial are interpreted as “different interpolation methods” although the same type of “spline interpolation” method is used. As another example, multiple interpolation data sets generated with the same type of interpolation method but the number of values to be interpolated during the same time period are different from each other are interpreted as “different interpolation methods” as well.

Subsequently, the processor 22 integrates the first interpolation data set IT1, the second interpolation data set IT2, and the third interpolation data set IT3 to generate integrated data IG. In addition, the processor 22 identifies, as a prediction result, a range RG defined by the maximum value MX and the minimum value MN among the multiple interpolated values included in the integrated data IG.

In general, the shorter the time interval for acquiring multiple observed values of the observed parameter, the less the necessity for interpolating an estimated value between the multiple observed values. As a result, it is possible to suppress degradation of the prediction model 23 that uses the time series data of the observed parameter as the input. On the other hand, there may be a case where it is difficult to ensure a sufficient number of time series data of a certain observed parameter like the axillary temperature in this example. However, according to the configuration of the present exemplary embodiment, a range defined by the maximum value and the minimum value selected from multiple interpolated values generated with multiple different methods is specified as a reasonable range within which the interpolated values may finally fall. As a result, it is possible to suppress a bias in the interpolation tendency that may be caused by relying on a specific interpolation method. Accordingly, it is possible to suppress degradation of prediction accuracy of a prediction model configured to use, as an input, a value interpolating observed values acquired in an environment in which time series data of an observed parameter are not stably acquired.

Particularly in the present exemplary embodiment, multiple interpolation data sets are generated with different types of interpolation methods. Accordingly, the above-described bias of the interpolation tendency can be further suppressed.

As illustrated in FIG. 1, the prediction system 1 may include an output device 3. The processor 22 of the prediction device 2 is configured to output, from the output interface 24, control data CT that causes the output device 3 to visualize the range within which the value predicted by the prediction model 23 for interpolating the observed values may fall. Examples of the output device 3 include a display device, a printing device, and the like. According to such a configuration, the user can confirm whether the predicted result visualized by the output device 3 is appropriate.

In this case, the output interface 24 is configured as a hardware interface adapted to output the control data CT. The control data CT may be in the form of analog data or digital data, in accordance with the specification of the output device 3. In the case where the control data CT is in the form of analog data, the output interface 24 includes an adequate conversion circuit including a D/A converter.

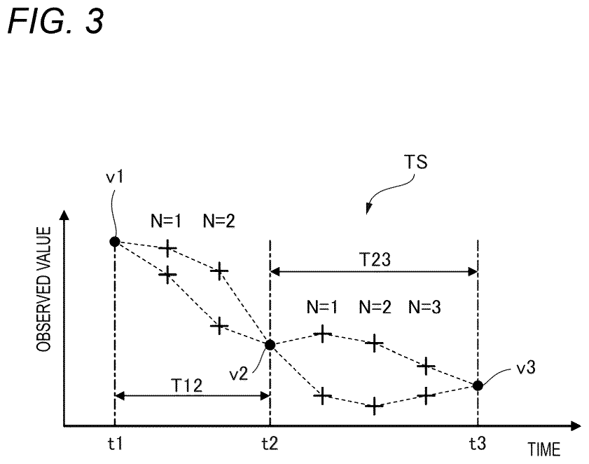

An example of the specific operation of the prediction model 23 will be described with reference to FIG. 3. In this example, the time series data TS received by the input interface 21 include an observed value v1 of the axillary temperature acquired at a time point t1, an observed value v2 of the axillary temperature acquired at a time point t2, and an observed value v3 of the axillary temperature acquired at the time point t3. In this example, the time interval for acquiring the observed values is not constant. In other words, a time interval T12 between the time points t1 and t2 is different from a time interval T23 between the time points t2 and t3.

The prediction device 2 according to the present example is configured to change the number of unobserved values to be interpolated so as to have a positive correlation with the time interval for acquiring the observed values. In other words, the longer the time interval for acquiring the observed values, the larger the number of unobserved values to be interpolated.

In the example illustrated in FIG. 3, two unobserved values are interpolated between the observed value v1 and the observed value v2 that are acquired with the time interval T12. On the other hand, three unobserved values are interpolated between the observed value v2 and the observed value v3 that are acquired with the time interval T23. The reference symbol N illustrated in FIG. 3 indicates the number of unobserved values to be interpolated.

As an example, the technique described with reference to FIG. 2 may be used for prediction of a reasonable range within which each interpolated value may fall. Namely, a range defined by the maximum value and the minimum value selected from multiple interpolated values generated with multiple different methods is specified as the prediction result.

As illustrated in FIG. 1, the prediction system 1 includes a prediction model generating device 4. The prediction model 23 of the prediction device 2 is generated through machine learning that is performed by the prediction model generating device 4. The prediction model generating device 4 includes an input interface 41, a processor 42, and an output interface 43. The prediction model generating device 4 is an example of a computing device.

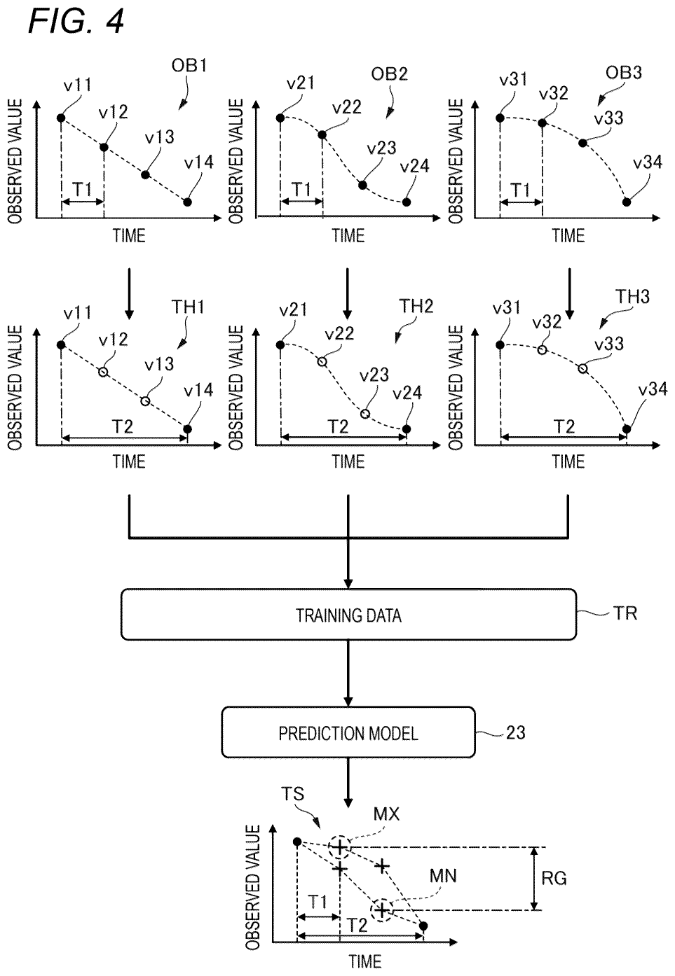

With reference to FIG. 4, a method of generating the prediction model 23 according to another example that is executed by the prediction model generating device 4 will be described.

The input interface 41 receives a first observation data set OB1 including multiple observed values of the axillary temperature acquired from a living body with a first time interval T1. The first observation data set OB1 includes four observed values v11 to v14.

The processor 42 generates a first downsampled data set TH1 by downsampling at least one of the multiple observed values included in the first observation data set OB1. In this example, the two observed values v12 and v13 acquired between the earliest observed value v11 and the latest observed value v14 are downsampled from the four observed values. As a result, the first downsampled data set TH1 includes multiple observed values at a second time interval T2 that is longer than the first time interval T1.

Similarly, the input interface 41 receives a second observation data set OB2 including multiple observed values of the axillary temperature acquired from the living body with the first time interval T1. The second observation data set OB2 includes four observed values v21 to v24.

Similarly to the case of the first observation data set OB1, the processor 42 generates a second downsampled data set TH2 by downsampling the two observed values v22 and v23 acquired between the earliest observed value v21 and the latest observed value v24 from the four observed values included in the second observation data set OB2. It should be noted that it is preferable that the observed values v21 and v24 left in the second downsampled data set TH2 are substantially equal to the observed values v11 and v14 left in the first downsampled data set TH1, respectively.

In this example, the input interface 41 additionally receives a third observation data set OB3 including multiple observed values acquired from the living body with the first time interval T1. The third observation data set OB3 includes four observed values v31 to v34.

Similarly to the case of the first observation data set OB1, the processor 42 generates a third downsampled data set TH3 by downsampling the two observed values v32 and v33 acquired between the earliest observed value v31 and the latest observed value v34 from the four observed values included in the third observation data set OB3. It should be noted that it is preferable that the observed values v31 and v34 left in the third downsampled data set TH3 are substantially equal to the observed values v11 and v14 left in the first downsampled data set TH1, respectively.

Subsequently, the processor 42 performs supervised learning with the first downsampled data set TH1 as the training data TR such that the downsampled observed values v12 and v13 are regarded as the ground truth. Similarly, the processor 42 performs supervised learning with the second downsampled data set TH2 as the training data TR such that the downsampled observed values v22 and v23 are regarded as the ground truth. Similarly, the processor 42 performs supervised learning with the third downsampled data set TH3 as the training data TR such that the downsampled observed values v32 and v33 are regarded as the ground truth.

Namely, the above machine learning causes a neural network to learn different changing patterns from one observed value to another observed value that may occur during the second time interval T2. The prediction model 23 generated by such machine learning is configured to predict, in response to the input of the time series data TS including two observed values acquired with the second time interval T2 from the subject S, a range within which each of two unobserved values to be interpolated between the two observed values with the first time interval T1 may fall. The prediction model 23 is configured to specify a maximum value MX and a minimum value MN among the multiple unobserved values that may be interpolated between the two observed values, and then output a range RG defined therebetween as a prediction result.

By performing the machine learning with the training data TR in which the second time interval T2 is so determined as to match with the time interval between multiple observed values included in the time series data TS acquired from the subject S, the prediction model 23 as generated can obtain capability of specifying, with at least one prediction, an appropriate range within which unobserved values to be interpolated between the observed values acquired with that time interval may fall. In a case where the time interval between the multiple observed values included in the time series data TS is not constant, machine learning with training data TR in which the second time interval T2 is varied may be performed to provide multiple prediction models 23. As a result, it is possible to configure a prediction device 2 capable of specifying, with at least one prediction, a range within which unobserved values to be interpolated between multiple observed values acquired with each of different time intervals may fall.

In general, in a case where a time interval for acquiring multiple observed values of an observed parameter is constant, a single prediction model can be uniquely applied to all time slots to perform prediction of a range within which unobserved values to be interpolated between the multiple observed values may fall. On the other hand, there may be a case where it is not constant the time interval for acquiring multiple observed values of a certain observed parameter like the axillary temperature in this example. However, according to the configuration of the present example, since the prediction of values to be interpolated is performed by selecting an adequate one of multiple prediction models generated by multiple machine learnings that are made different in correspondence with the time interval, it is possible to suppress degradation of the prediction accuracy of the prediction model that uses, as an input, values interpolated between observed values in the case where the time interval for acquiring the observed values is not constant.

Multiple sets of training data TR in which the second time interval T2 is made different from each other are preferably generated by changing the interval of downsampling from the same observation data set. For example, by downsampling the observed value v12 and the observed value v14 out of the observed values v11 to v14 included in the first observation data set OB1, it is possible to generate another set of training data TR in which the second time interval T2 is made shorter than the first downsampled data set TH1.

According to such a configuration, it is possible to generate a prediction model capable of being adapted to a case where the time interval between the multiple observed values included in the time series data TS is not constant, efficiently from a data set including a limited number of observed values.

Each of the processor 22 of the prediction device 2 and the processor 42 of the prediction model generating device 4 having various functions described above may be implemented by one or more non-exclusive microprocessors configured to cooperate with one or more non-exclusive memories. Examples of the non-exclusive microprocessor include a CPU, an MPU, and a GPU. Examples of the non-exclusive memory include a ROM and a RAM. In this case, a computer program for executing the above-described processing may be stored in the ROM. The ROM is an example of a non-transitory computer-readable medium having stored a computer program. The non-exclusive microprocessor designates at least a part of the program stored in the ROM, loads the designated program in the RAM, and executes the above-described processing in cooperation with the RAM. The computer program may be pre-installed in a non-exclusive memory or may be downloaded from an external server with a communication network, and then installed in the non-exclusive memory. In this case, the external server is an example of a non-transitory computer readable medium having stored a computer program.

Each of the processor 22 and the processor 42 may be implemented by one or more exclusive integrated circuitries capable of executing the above-described computer program. Examples of the exclusive integrated circuitry include a microcontroller, an ASIC, and an FPGA. In this case, the above-described computer program is pre-installed in a memory element included in the exclusive integrated circuitry. The memory element is an example of a non-transitory computer-readable medium having stored a computer program. Each of the processor 22 and the processor 42 may also be implemented by a combination of the non-exclusive microprocessor and the exclusive integrated circuitry.

Each of the configurations exemplified above is merely illustrative for facilitating understanding of the presently disclosed subject matter. Each exemplary configuration may be appropriately modified or combined with another exemplary configuration within the scope of the presently disclosed subject matter.

In the above exemplary embodiment, the prediction device 2 and the prediction model generating device 4 are illustrated as devices independent of each other. In this case, the input interface 21 and the output interface 43 are configured as hardware interfaces. However, both devices may be implemented as different functional modules in a single device. In this case, both interfaces may be implemented as software interfaces.

The prediction model 23 need not necessarily be installed in the prediction device 2. Although not illustrated, the prediction device 2 may be connected to an external server device such that communication with the external server device via a communication network is enabled. In this case, the prediction model 23 may be installed in the external server device.

In the above exemplary embodiment, the prediction model generating device 4 generates the prediction model 23 through the machine learning using the neural network. However, the prediction model 23 may be generated through other machine learning algorithms. Examples of such machine learning algorithms include linear regression, logistic regression, decision trees, random forests, support vector machines, and the like.

As used herein, the meaning of the term “prediction” may be interpreted so as not to exclude the meaning of each of the terms “estimation” and “forecasting”.

1. A prediction device, comprising:
an interface configured to receive time series data including multiple observed values of an observed parameter that are acquired at different time points for obtaining physiological information of a subject; and
a processor configured to input the time series data into a prediction model to output, as a prediction result, a range within which values interpolating the multiple observed values may fall,
wherein the processor is further configured to:
generate at least one first interpolated value by interpolating the multiple observed values with a first method;
generate at least one second interpolated value by interpolating the multiple observed values with a second method that is different from the first method; and
identify, as the prediction result, a range defined between a maximum value and a minimum value among the first interpolated value and the second interpolated value.

2. The prediction device according to claim 1,
wherein the first method and the second method differ in a type of interpolating method.

3. The prediction device according to claim 1,
wherein the processor is configured to cause an output device to visualize the range.

4. A prediction device, comprising:
an interface configured to receive time series data including multiple observed values of an observed parameter that are acquired at different time points for obtaining physiological information of a subject; and
a processor configured to input the time series data into a prediction model to perform at least one prediction of a range within which values interpolating the multiple observed values may fall,
wherein the processor is further configured to select, as the prediction model, one of multiple prediction models in accordance with a time interval between the different time points; and
wherein the multiple prediction models are generated by machine learning so as to correspond to different time intervals between the different time points.

5. The prediction device according to claim 4,
wherein the processor is configured to cause an output device to visualize the range.

6. A method of generating a prediction model adapted to predict physiological information with a computing device, the method comprising:
acquiring observation data including multiple observed values of an observed parameter acquired from a living body with a first time interval for obtaining the physiological information;
downsampling at least one of the multiple observed values to generate training data including multiple observed values provided with a second time interval that is longer than the first time interval;
performing supervised learning with the training data such that the multiple observed values as downsampled are regarded as a ground truth;
configuring the prediction model so as to predict, with respect to multiple observed values of the observed parameter acquired from a living body with the second time interval as an input, a range within which at least one value interpolating the multiple observed values as inputted may fall; and
generating another training data from the observation data while changing the second time interval.

7. A computing device configured to generate a prediction model adapted to predict physiological information, the computing device comprising:
an interface configured to receive observation data including multiple observed values of an observed parameter acquired from a living body with a first time interval for obtaining the physiological information; and
a processor configured to:
downsample at least one of the multiple observed values to generate training data including multiple observed values provided with a second time interval that is longer than the first time interval;
perform supervised learning with the training data such that the multiple observed values as downsample are regarded as a ground truth;
configure the prediction model so as to predict, with respect to multiple observed values of the observed parameter acquired from a living body with the second time interval as an input, a range within which at least one value interpolating the multiple observed values as inputted may fall; and
generate another training data from the observation data while changing the second time interval.
